(19) United States
(12) Patent Application Publication
(10) Pub. No.: US 2023/0255806 A1
(43) Pub. Date: Aug. 17, 2023

(54) VAGINAL STENT

(71) Applicant: BioTex, Inc., Houston, TX (US)

(72) Inventors: JESSICA PHILLIPS, Houston, TX (US); KIMBERLY NEELY, Houston, TX (US); JULIE CATHERINE-ELISE HAKIM, Houston, TX (US); ANISHAA POTNIS, Houston, TX (US); ASHOK GOWDA, Houston, TX (US)

(21) Appl. No.: 18/017,298

(22) PCT Filed: Jul. 23, 2021
(86) PCT No.: PCT/US2021/043068

§ 371 Date: Jan. 20, 2023

Related U.S. Application Data
(60) Provisional application No. 63/055,680, filed on Jul. 23, 2020.

(51) Int. Cl. A61F 2/94

(52) U.S. Cl. CPC A61F 2/94; A61F 2230/005

(57) ABSTRACT

This invention relates to methods and devices for maintaining the opening and/or caliber of a bodily orifice, particularly to methods and devices for maintaining the opening and/or caliber of the vaginal cavity of a human, and more particularly to methods and devices for maintaining the opening and/or caliber of the vaginal cavity of a human with an inserted vaginal stent. In general, a device for maintaining the opening and/or caliber of a bodily orifice, such as a human vaginal cavity, may include a vaginal stent shaped and having material properties for insertion into the bodily orifice and holding the bodily orifice open and/or at a given caliber, such as to resist collapse or closure of the bodily orifice. The vaginal stent may further include features for facilitating placement, retention in the bodily orifice, size or shape adjustment, drainage and/or removal of the vaginal stent after use.

### CROSS-REFERENCE TO RELATED APPLICATIONS

This Patent Cooperation Treaty international application claims the benefit and priority of U.S. provisional patent application Ser. No. 63/055,680, filed Jul. 23, 2020, entitled “VAGINAL STENT”, the contents of which is hereby incorporated by reference in its entirety.

### FIELD OF THE INVENTION

This invention relates to methods and devices for maintaining the opening and/or caliber of a bodily orifice, particularly to methods and devices for maintaining the opening and/or caliber of the vaginal cavity of a human, and more particularly to methods and devices for maintaining the opening and/or caliber of the vaginal cavity of a human with an inserted vaginal stent.

### BACKGROUND OF THE INVENTION

The vaginal stent device is intended to maintain the caliber of the vaginal canal and/or cavity following medical procedures such as gynecological procedures, cancer/radiological treatment or surgical procedures to restore, enlarge, or create a vagina in adolescents or adults.

### SUMMARY OF THE INVENTION

This invention relates to methods and devices for maintaining the opening and/or caliber of a bodily orifice, particularly to methods and devices for maintaining the opening and/or caliber of the vaginal cavity of a human, and more particularly to methods and devices for maintaining the opening and/or caliber of the vaginal cavity of a human with an inserted vaginal stent.

In general, a device for maintaining the opening and/or caliber of a bodily orifice, such as a human vaginal cavity, may include a vaginal stent shaped and having material properties for insertion into the bodily orifice and holding the bodily orifice open and/or at a given caliber, such as to resist collapse or closure of the bodily orifice. The vaginal stent may further include features for facilitating placement, retention in the bodily orifice, size or shape adjustment, drainage and/or removal of the vaginal stent after use.

In a first aspect of the invention, the vaginal stent includes a body with a widened retaining portion for retention in a bodily cavity, such as in the vaginal cavity by support of the pubic bone, and a stem portion for retention in the canal of a bodily orifice, such as the vaginal canal. In any of the embodiments of the invention, the body may generally be formed from a flexible, collapsible and/or elastic material such that it may be reversibly deformed for insertion and removal from the bodily cavity.

In a second aspect of the invention, the vaginal stent may utilize a retention mechanism to provide a retention action against the walls of the bodily orifice, such as by altering the size or shape of the stem portion of the body or through other retention forces, such as suction against the walls of the bodily orifice. In any of the embodiments of the invention, the retention mechanism may generally press against the walls of the bodily orifice to provide a retaining force for the vaginal stent. The retention mechanism may utilize the size of the stem portion to press against the walls of the bodily orifice. The retention mechanism may also utilize a portion that changes in size after insertion of the vaginal stent, such as a balloon, an insert for the stem portion, spring-like or elastic expansion portions or features, and/or any other appropriate mechanism. The retention mechanism may further include features for actuating the retention action of the vaginal stent to enable insertion and removal from the bodily orifice.

In a third aspect of the invention, the vaginal stent may include features for providing drainage from the bodily cavity. In any of the embodiments of the invention, the body of the vaginal stent may include lumens, drainage holes, channels, apertures, and/or any other appropriate drainage features or combinations thereof. This may be generally desirable to prevent the buildup of fluids in the bodily cavity during use of the device.

In a fourth aspect of the invention, the vaginal stent may be placed with an applicator to aid in easy placement in a bodily orifice. In any of the embodiments of the invention, the body of the vaginal stent may be retained in an applicator, which may then be placed in a bodily orifice and withdrawn to remove the applicator and leave the vaginal stent in place. The applicator may, for example, serve to deform, compress or otherwise hold the body of the vaginal stent in a smaller or more compact configuration to ease insertion. The applicator may also hold or otherwise prevent the retention mechanism from engaging the bodily cavity during insertion.

The present invention together with the above and other advantages may best be understood from the following detailed description of the embodiments of the invention and as illustrated in the drawings. The following description, while indicating various embodiments of the invention and numerous specific details thereof, is given by way of illustration and not of limitation. Many substitutions, modifications, additions or rearrangements may be made within the scope of the invention, and the invention includes all such substitutions, modifications, additions or rearrangements.

### BRIEF DESCRIPTION OF THE FIGURES

FIGS. 1, 1a, 2, 2a, 2b, 2c, 2d and 2e illustrate embodiments of a vaginal stent with a retaining portions and stem portions;

FIGS. 3, 3a, 3b, 3c and 3d illustrate insertion and removal of embodiments of a vaginal stent; and

FIGS. 4, 5, 5a, 6, 6a, 7, 7a, 8, 8a, 9, 9a, 10, 10a, 11, 11a, 12, 13, 13a, 13b, 13c and 13d illustrate different embodiments of vaginal stents with different retaining portions and retention features.

### DETAILED DESCRIPTION OF THE INVENTION

The detailed description set forth below is intended as a description of the presently exemplified systems, devices and methods provided in accordance with aspects of the present invention and are not intended to represent the only forms in which the present invention may be prepared or utilized. It is to be understood, rather, that the same or equivalent functions and components may be accomplished by different embodiments that are also intended to be encompassed within the spirit and scope of the invention. Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the exemplary methods, devices and materials are now described. All publications mentioned herein are incorporated herein by reference for the purpose of describing and disclosing, for example, the designs and methodologies that are described in the publications which might be used in connection with the presently described invention. The publications listed or discussed above, below and throughout the text are provided solely for their disclosure prior to the filing date of the present application. Nothing herein is to be construed as an admission that the inventors are not entitled to antedate such disclosure by virtue of prior invention.

This invention relates to methods and devices for maintaining the opening and/or caliber of a bodily orifice, particularly to methods and devices for maintaining the opening and/or caliber of the vaginal cavity of a human, and more particularly to methods and devices for maintaining the opening and/or caliber of the vaginal cavity of a human with an inserted vaginal stent. In general, a device for maintaining the opening and/or caliber of a bodily orifice, such as a human vaginal cavity, may include a vaginal stent shaped and having material properties for insertion into the bodily orifice and holding the bodily orifice open and/or at a given caliber, such as to resist collapse or closure of the bodily orifice. The vaginal stent may further include features for facilitating placement, retention in the bodily orifice, size or shape adjustment, drainage and/or removal of the vaginal stent after use.

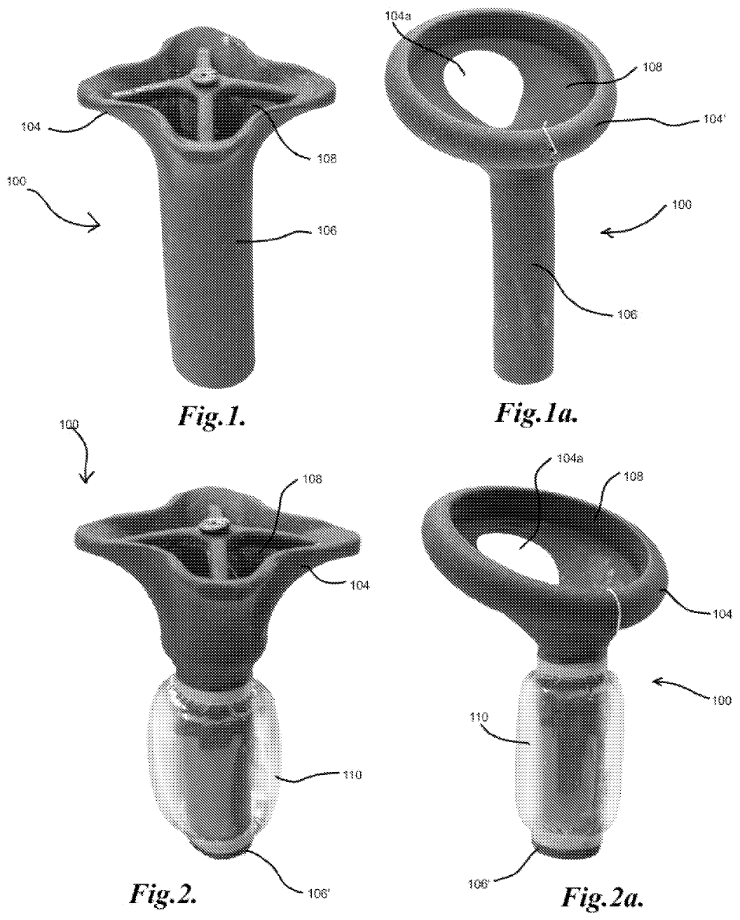

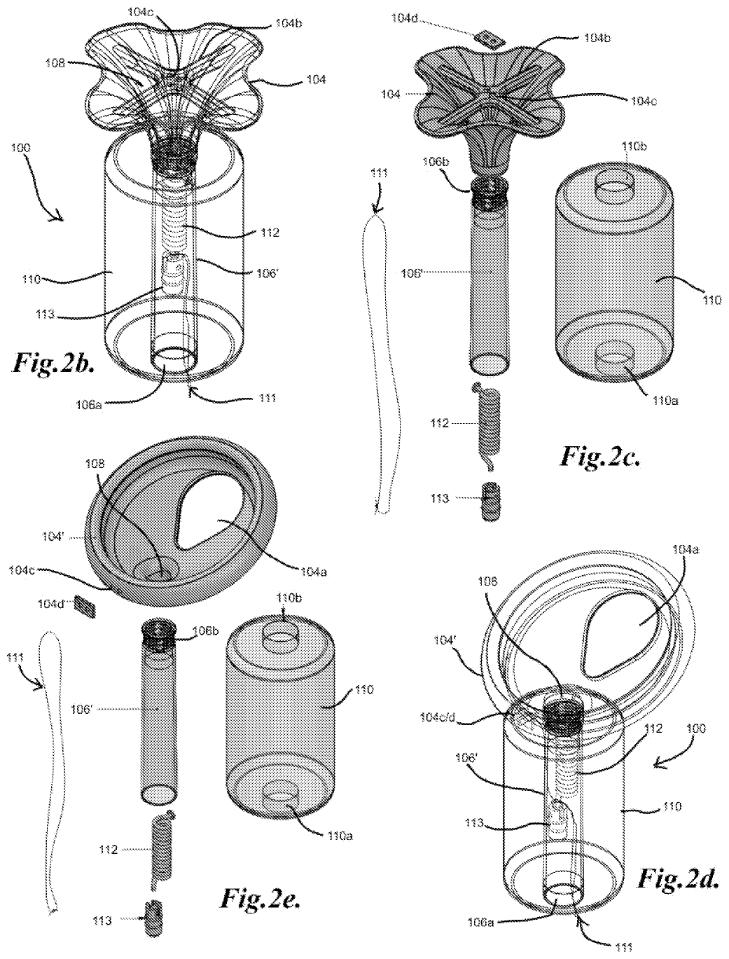

In a first aspect of the invention, the vaginal stent, as illustrated with the vaginal stent 100 in FIGS. 1, 1a, 2 and 2a, includes a body 102 with a widened retaining portion, as shown with the retaining portions 104 and 104′, for retention in a bodily cavity, such as in the vaginal cavity by support of the pubic bone, and a stem portion for retention in the canal of a bodily orifice, such as the vaginal canal, as illustrated with stems 106 and 106′. In any of the embodiments of the invention, the body 102 may generally be formed from a flexible, collapsible and/or elastic material such that it may be reversibly deformed for insertion and removal from the bodily cavity. In any of the embodiments of the invention, the material of the body 102 may include natural or synthetic polymeric elastomers, such as silicone, natural rubber, synthetic rubber (e.g. styrene butadiene, polyisoprene, polychloroprene a.k.a. neoprene, etc.), and/or any other appropriate elastomer or combination thereof. In general, the material may be selected for biocompatibility or non-reactivity in medical settings, ability to be sterilized (e.g. by autoclave, ethylene oxide, ultraviolet, etc.).

In any of the embodiments of the invention, the body 102 may also include multiple materials or regions of different physical properties. This may be desirable to impart enhanced physical characteristics to different portions of the body 102. For example, an inner core or support structure within the body 102 may utilize a harder, stiffer or more rigid material, such as to provide structural support to the body 102 and/or to enhance the opening/collapse-resistance of the vaginal stent 100 against the bodily orifice/cavity. Examples of core or support materials may include, but are not limited to, polyetheretherketone (PEEK), polysulfone, polycarbonate, and/or any other appropriate material. Additionally, the flexible, collapsible and/or elastic material may be overmolded or coated onto the core or support to provide the deformation properties discussed above, such as by overmolding silicone or another material onto the core or support.

In another example, the body 102 may include regions of the same material with different hardness (e.g. durometer) to provide variations in the physical characteristics in different portions. For example, the stems 106, 106′ may include a higher durometer material than the retaining portions 104, 104′ to provide additional support against collapse of the bodily cavity, while a lower durometer material in the retaining portions 104, 104′ may be utilized to aid the insertion and removal of the vaginal stent 100 by allowing the retaining portions 104, 104′ to collapse or deform more easily.

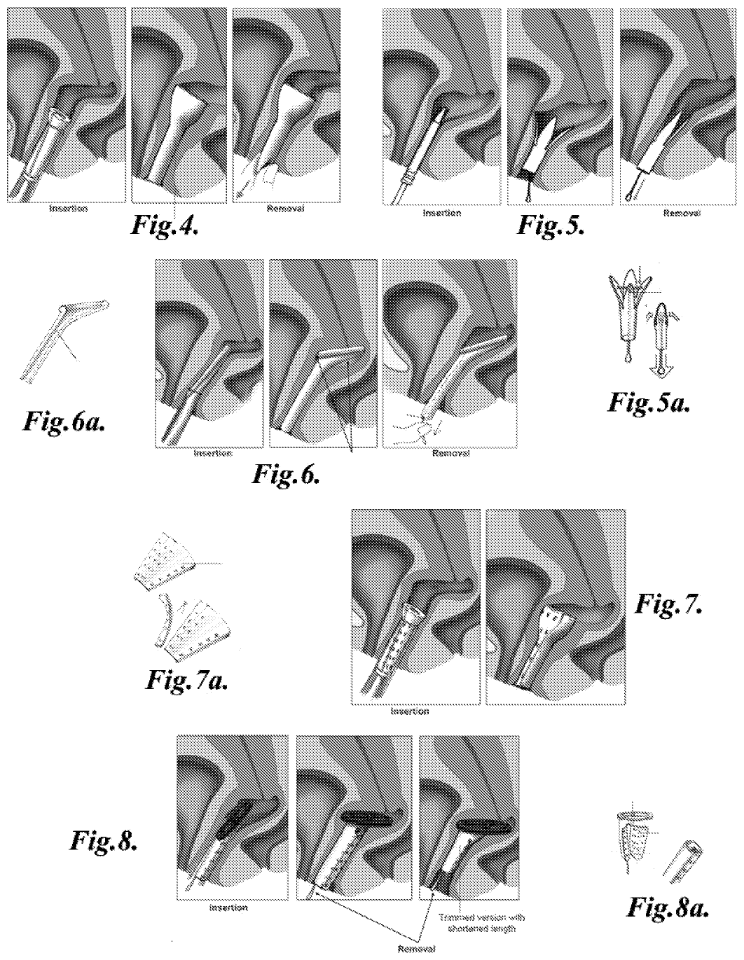

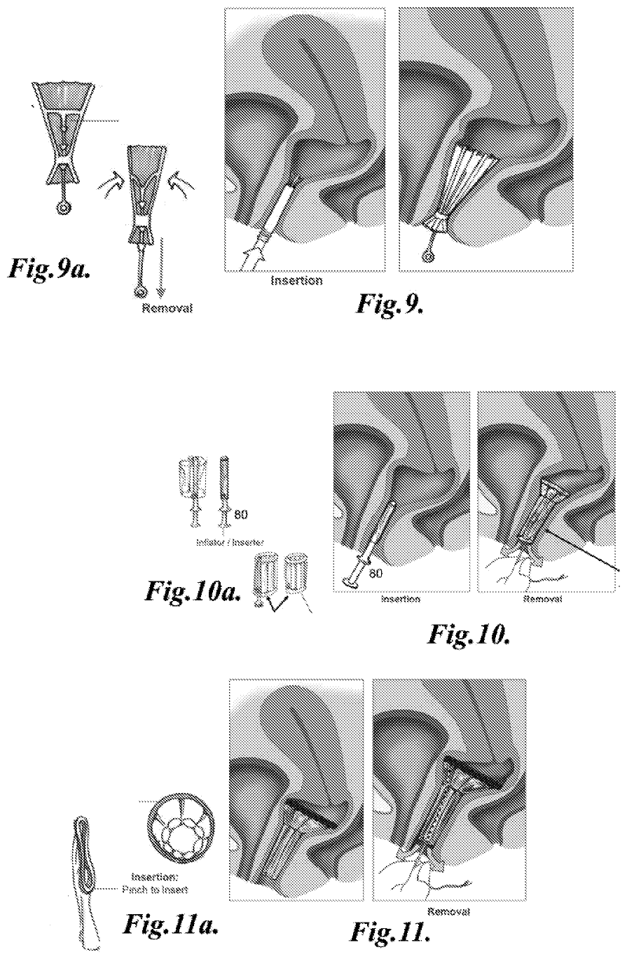

In any of the embodiments of the invention, the retaining portions 104, 104′ may be shaped to be held in place in the bodily cavity by an anatomical feature, such as by the pubic bone when inserted into the vaginal cavity, as illustrated with the cone-like or flower-like retaining portion 104 in FIGS. 1 and 2, or the disc-like retaining portion 104′ in FIGS. 1a and 2a. Other shapes may also be utilized, such as the funnel-like cone shape in FIG. 4, the flower-like shape in FIGS. 5 and 5a (the petals may be retracted by the illustrated pull actuator for insertion and/or removal), the disc-like shape in FIGS. 6 and 6a, the roll-up funnel-like cone shape in FIGS. 7 and 7a, the ring-like shape in FIGS. 8 and 8a, the umbrella-like shape in FIGS. 9 and 9a, and/or any other appropriate shape. Cutouts, gaps, and/or other negative spaces may be utilized to aid increasing the deformability of the retaining portion 104, 104′, as shown with the gaps between the “petals” of the flower-like shapes or the cutout 104a in FIGS. 1, 1a, 2 and 2a.

The size of the body 102 may also be adjustable or customizable. In general, the stem portions, such as the stem 106, may be cut or trimmed in length such that excess material does not protrude from the bodily orifice after insertion. FIGS. 7, 7a, 8 and 8a illustrate an example of an adjustable body 102 where material may be cut off prior to rolling up to form a smaller body 102 (e.g. with the illustrated snap features). Different versions of the vaginal stent 100 may also be produced at different sizes, such as to accommodate different bodily orifices/cavities or for different aged/sized patients (e.g. children, teenagers, adults, older patients, etc.).

The vaginal stent may further include a feature for aiding removal, such as a handle, pull string, wire or other appropriate feature that may be pulled to aid in removal from the bodily cavity, as illustrated with the pull strings in the various figures. The pull string, such as the pull string 111 illustrated in FIGS. 2b, 2c, 2d and 2e, may generally attach to a portion of the body 102, such as at a portion 104c of the retaining portions 104, 104′ such that, for example, the pulling force may be exerted on the larger portion of the vaginal stent 100, such as on the reinforcing bars 104b in FIGS. 2b and 2c or on the disc-like portion 104′ in FIGS. 2d and 2e. The pull string 111 may also be useful in applying force onto the retaining portions 104, 104′ during removal, such as to cause the material to fold, compress, collapse or otherwise deform for a smaller size for easier removal. The pull string 111 may be, for example, fed through apertures or holes in the material of the body 102, as illustrated with portion 104c. The portion 104c may also be reinforced such that the material may better resist the pulling force during removal, such as through a reinforcement plate 104d which may be attached or disposed on the body 102, or it may be co-molded into the material. The reinforcement plate 104d may, for example, be made from metal, rigid plastic, etc.

In a second aspect of the invention, the vaginal stent may utilize a retention mechanism to provide a retention action against the walls of the bodily orifice, such as by altering the size or shape of the stem portion of the body or through other retention forces, such as suction against the walls of the bodily orifice. In any of the embodiments of the invention, the retention mechanism may generally press against the walls of the bodily orifice to provide a retaining force for the vaginal stent. The retention mechanism may utilize the size of the stem portion to press against the walls of the bodily orifice. The retention mechanism may also utilize a portion that changes in size after insertion of the vaginal stent, such as a balloon, an insert for the stem portion, spring-like or elastic expansion portions or features, and/or any other appropriate mechanism. The retention mechanism may further include features for actuating the retention action of the vaginal stent to enable insertion and removal from the bodily orifice.

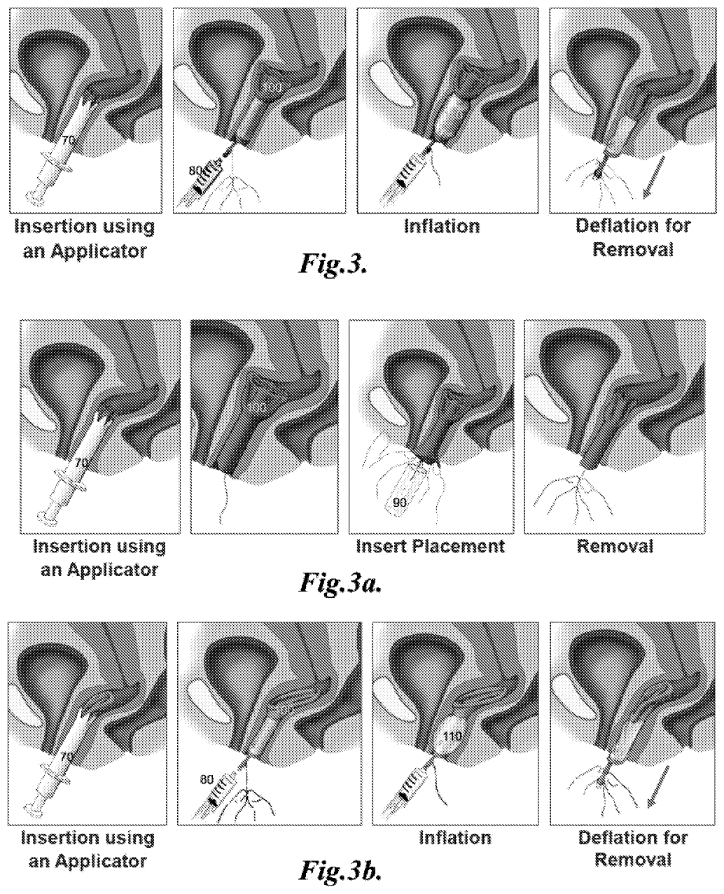

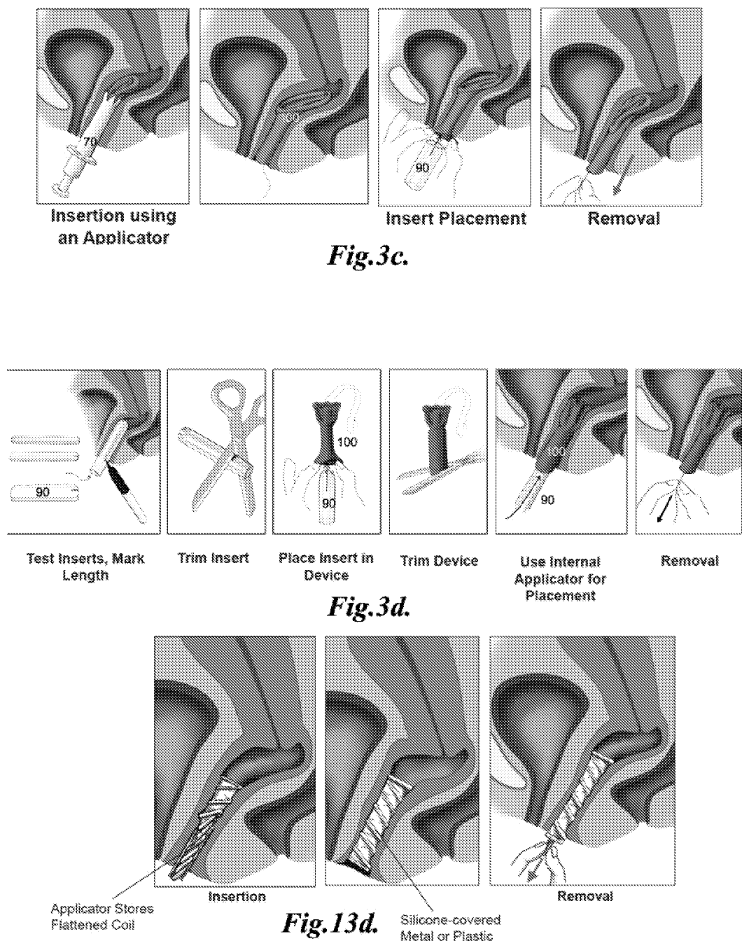

In some embodiments, such as illustrated in FIGS. 2, 2a, 2b, 2c, 2d and 2e, a retention mechanism includes an inflatable balloon 110 about the stem 106′ which may be inflated to retain the vaginal stent 100 in a bodily orifice, such as illustrated with the vaginal canal in FIGS. 3 and 3b. The inflatable balloon 110 may generally include a valve or other connection such that an inflator 80 may be connected to inflate and/or to deflate the inflatable balloon 110 when the vaginal stent 100 is to be removed, as illustrated with the valve body 113 and the connector conduit 112 which may interface with the inflatable balloon 110, such as through an interface 106b on the stem 106′, as illustrated in FIGS. 2b, 2c, 2d and 2e. In some embodiments, all or a majority of the body 102 may be formed into a balloon, as illustrated with inflatable bodies 102 in FIGS. 10, 10a, 11 and 11a. The inflator 80 and/or the inflatable portions of any of the embodiments may further include a coupling interface, such as a magnetic coupling, a threaded coupling, or other appropriate connection. Inflatable components may also be made of any appropriate material that may deform upon inflation/deflation, such as, for example, silicone, pellethane, polyurethane, multiple layers of materials, etc. The inflatable portions may further be shaped to enhance their retention characteristics, such as with ribs or contouring surfaces to better engage the walls of the bodily orifice. The degree of inflation may also be utilized to adjust for the size and fit of the inflatable portion in the bodily orifice, as higher inflation may generally produce a larger inflatable portion and/or greater retention force. The inflatable balloon 110 may, for example, be formed as a cylindrical or tube-like membrane, as shown in the exploded views of FIGS. 2c and 2e, where portions 110a and 110b may form seals against the stem 106′ to enclose the inflating volume.

In some embodiments, the inflatable portions may be connected to a conduit or other member that may be pulled out of the lumen 106a for easier access and connection to an inflator or for deflation. The conduit or other member may, for example, be elastic, spring-like or otherwise able to be pulled out and retractable into the lumen 106a such that it does not hang out of the bodily orifice when not being accessed for inflation/deflation. FIGS. 2b, 2c, 2d and 2e illustrate a retractable conduit as a coiled plastic tubing 112 which retains itself in the lumen 106a and may be pulled out, such as via the pull string 111 which may be attached to the tubing 112 as shown (near the valve body 113), to be interfaced with an inflator 80, such as by connecting a syringe or pump to the valve body 113. An extending connector, such as additional tubing, may also be connected between the inflator 80 and the valve body 113, for example, to increase spacing from the body orifice, such as for ease of access. The coiled plastic of the tubing 112 may then be released, such that the elastic or retracting force of the material/shape of the tubing 112 may pull it back into the lumen 106a.

In some embodiments, as illustrated in FIGS. 3a and 3c, inserts 90 may be inserted into the stem 106 to stretch and widen the stem 106 after placement into the bodily orifice. Depending on the bodily orifice and the user, the insert 90 and/or portions of the body 102 may also be trimmed or otherwise adjusted to provide the desired insertion depth without excess material protruding from the bodily orifice, as illustrated in FIG. 3d.

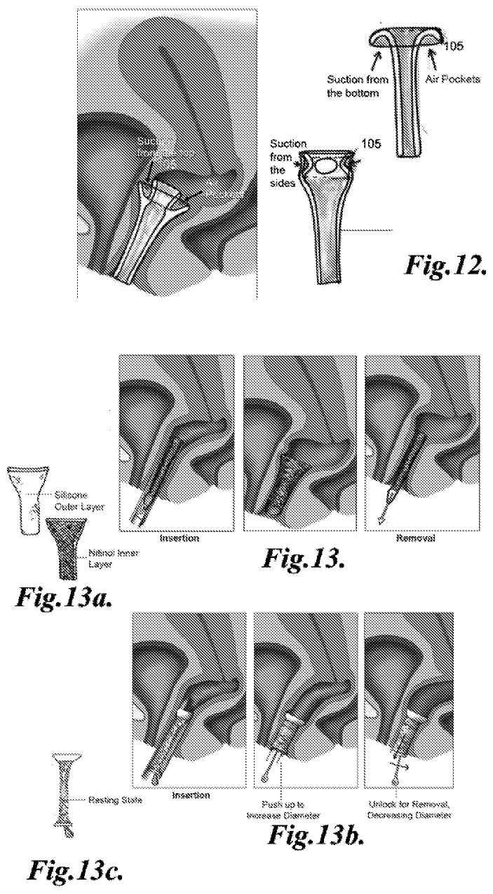

In some embodiments, portions of the body 102 may be utilized to retain the vaginal stent 100 in the bodily cavity through suction against the walls of the bodily cavity, as illustrated with the suction generating portions 105 for forming air pockets to hold onto the walls of the vaginal cavity by suction in FIG. 12.

In some embodiments, the body 102 may include spring-like elements that may be utilized to reversibly alter the shape of the body 102 as a retention mechanism. FIGS. 13, 13a, 13b, 13c and 13d illustrate examples of spring-like elements that cause the body 102 to push against the walls of the bodily orifice. FIGS. 13 and 13a illustrate an embedded retention mechanism similar to a “finger trap” and utilizing a spring-like mesh or shape-memory alloy (e.g. nitinol) such that the embedded retention mechanism pushes the body 102 against the walls of the bodily orifice when not subjected to a stressed state (e.g. by being pulled to deform in the insertion and removal steps). FIGS. 13b and 13c illustrate a similar mechanism, but where the embedded retention mechanism is stressed by pushing in an actuator to retain in the bodily orifice and released to shrink back to a relaxed state. FIG. 13d illustrates the use of a coiled spring (e.g. helical spring, coil spring) to push outward on the walls of the bodily orifice for retention of the body 102.

In a third aspect of the invention, the vaginal stent may include features for providing drainage from the bodily cavity. In any of the embodiments of the invention, the body of the vaginal stent may include lumens, drainage holes, channels, apertures, and/or any other appropriate drainage features or combinations thereof. This may be generally desirable to prevent the buildup of fluids in the bodily cavity during use of the device. FIGS. 1, 1a, 2, 2a, 2b, 2c, 2d and 2e illustrate apertures 108 which connect to an internal lumen 106a in the stem 106, 106′ to provide drainage of fluid. In embodiments using an insert 90, the insert 90 may also have an internal lumen, channels, apertures or other drainage features to enable fluid to drain while the insert 90 is within the stem 106.

In a fourth aspect of the invention, the vaginal stent may be placed with an applicator to aid in easy placement in a bodily orifice. In any of the embodiments of the invention, the body of the vaginal stent may be retained in an applicator, which may then be placed in a bodily orifice and withdrawn to remove the applicator and leave the vaginal stent in place. The applicator may, for example, serve to deform, compress or otherwise hold the body of the vaginal stent in a smaller or more compact configuration to ease insertion. The applicator may also hold or otherwise prevent the retention mechanism from engaging the bodily cavity during insertion. In general, the applicator may take the form of an elongated tube, as shown in the figures with applicator 70, to hold the vaginal stent 100 within for insertion into a bodily orifice/cavity. The applicator 70 may further include a plunger or other feature to push the vaginal stent 100 out of the applicator 70 as the applicator 70 is withdrawn to leave the vaginal stent 100 within the bodily orifice/cavity. In embodiments utilizing an inflatable portion, such as the inflatable balloon 110, the applicator 70 may also include an inflator 80 as part of the applicator 70.

Although the invention has been described with respect to specific embodiments thereof, these embodiments are merely illustrative, and not restrictive of the invention. The description herein of illustrated embodiments of the invention, including the description in the Abstract and Summary, is not intended to be exhaustive or to limit the invention to the precise forms disclosed herein. Rather, the description is intended to describe illustrative embodiments, features and functions in order to provide a person of ordinary skill in the art context to understand the invention without limiting the invention to any particularly described embodiment, feature or function, including any such embodiment feature or function described in the Abstract or Summary. While specific embodiments of, and examples for, the invention are described herein for illustrative purposes only, various equivalent modifications are possible within the spirit and scope of the invention, as those skilled in the relevant art will recognize and appreciate. As indicated, these modifications may be made to the invention in light of the foregoing description of illustrated embodiments of the invention and are to be included within the spirit and scope of the invention. Thus, while the invention has been described herein with reference to particular embodiments thereof, a latitude of modification, various changes and substitutions are intended in the foregoing disclosures, and it will be appreciated that in some instances some features of embodiments of the invention will be employed without a corresponding use of other features without departing from the scope and spirit of the invention as set forth. Therefore, many modifications may be made to adapt a particular situation or material to the essential scope and spirit of the invention.

Reference throughout this specification to “one embodiment”, “an embodiment”, or “a specific embodiment” or similar terminology means that a particular feature, structure, or characteristic described in connection with the embodiment is included in at least one embodiment and may not necessarily be present in all embodiments. Thus, respective appearances of the phrases “in one embodiment”, “in an embodiment”, or “in a specific embodiment” or similar terminology in various places throughout this specification are not necessarily referring to the same embodiment. Furthermore, the particular features, structures, or characteristics of any particular embodiment may be combined in any suitable manner with one or more other embodiments. It is to be understood that other variations and modifications of the embodiments described and illustrated herein are possible in light of the teachings herein and are to be considered as part of the spirit and scope of the invention.

In the description herein, numerous specific details are provided, such as examples of components and/or methods, to provide a thorough understanding of embodiments of the invention. One skilled in the relevant art will recognize, however, that an embodiment may be able to be practiced without one or more of the specific details, or with other apparatus, systems, assemblies, methods, components, materials, parts, and/or the like. In other instances, well-known structures, components, systems, materials, or operations are not specifically shown or described in detail to avoid obscuring aspects of embodiments of the invention. While the invention may be illustrated by using a particular embodiment, this is not and does not limit the invention to any particular embodiment and a person of ordinary skill in the art will recognize that additional embodiments are readily understandable and are a part of this invention. As used herein, the terms “comprises,” “comprising,” “includes,” “including,” “has,” “having,” or any other variation thereof, are intended to cover a non-exclusive inclusion. For example, a process, product, article, or apparatus that comprises a list of elements is not necessarily limited only those elements but may include other elements not expressly listed or inherent to such process, process, article, or apparatus.

1. A device for retaining the caliber of a bodily orifice comprising:
a body having a widened retaining portion and a stem portion;
at least one drainage aperture in said body; and
a retaining feature adapted to reversibly deform at least a portion of said body to engage and bias against a wall of a bodily orifice.

2. The device of claim 1, wherein said widened retaining portion is selected from the group consisting of cone-like, flower-like and disc-like shapes.

3. The device of claim 1, further comprising a dilating feature for enlarging said stem portion.

4. The device of claim 3, wherein said dilating feature is selected from the group consisting of an inflatable balloon, an expanding coil, a shape memory section, a spring section and a dilating insert.

5. The device of claim 1, further comprising a removal feature adapted to deform said widened retaining portion to assist in removal of said device from said bodily orifice.

6. The device of claim 5, wherein said removal feature comprises a pull string.

7. The device of claim 4, further comprising a feature for actuating said dilating feature.

8. The device of claim 3, wherein said dilating feature is an inflatable balloon and further comprises an inflator for inflating said inflatable balloon.

9. The device of claim 1, further comprising an applicator for inserting said device into said bodily orifice.

10. The device of claim 4, wherein said dilating feature is disposed about said stem portion.

11. The device of claim 1, wherein said drainage aperture comprises an opening in said retaining portion.

12. The device of claim 1, wherein said bodily orifice is a vaginal cavity of a human.

13. The device of claim 1, wherein said body comprises a material selected from the group consisting of polyetheretherketone (PEEK), polysulfone, polycarbonate, and/or any other appropriate material.
